(19) United States
(12) Patent Application Publication
(10) Pub. No.: US 2009/0216194 A1
(43) Pub. Date: Aug. 27, 2009

(54) Medical Device Adapted for Detection of Drug Condition

(76) Inventors: Per Erik Elgård Pedersen, Haslev (DK); John Stern Nielsen, Allerod (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/661,868

(22) PCT Filed: Sep. 1, 2005
(86) PCT No.: PCT/EP2005/054294

§ 371 Date: May 6, 2009

(30) Foreign Application Priority Data
Sep. 2, 2004 (DK) PA 2004 01327
Sep. 2, 2004 (DK) PA 2004 01328

(51) Int. Cl. A61M 5/142

(52) U.S. Cl. 604/151

(57) ABSTRACT

A drug delivery device is provided, comprising a reservoir adapted to contain a liquid drug, an outlet, and expelling means for, in a situation of use, expelling a drug from the reservoir via the outlet. The device further comprises a light source (575) adapted for directing a beam of light through the drug, and detection means (576, 577) allowing a transmission characteristic of the light beam through the drug to be detected. The detecting means may be either in form of a transparent area allowing a user to inspect a portion of the device through which the light beam is directed, or light detection means used in combination with an electronic controller.

The present invention generally relates to drug delivery devices comprising means for checking a condition of a drug to be delivered, especially, but not restricted to, fibrillation of insulin.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a hollow infusion needle, as well as expelling means for expelling a drug out of the reservoir and through the skin of the subject via the hollow needle. The delivery device may be adapted for discrete use, i.e. injection of an amount of a drug a given number of times during the day, or they may be adapted for continuous or quasi-continuous delivery of drug through a permanent fluid connection between the delivery device and the patient. The former type of device is often referred to as a pen device and the latter type is often termed an infusion pump.

A “pen” is typically a mechanical pen-formed device, however, it may have any desirable configuration just as it may comprise a motor for assisted injection of drug.

Basically, infusion pumps can be divided into two classes. The first class comprises durable infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals. Examples of this type of pump are shown in U.S. Pat. Nos. 4,562,751 and 4,685,903 hereby incorporated by reference.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniences, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump (also know as a bleeding hole pump)), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

The disposable pumps generally comprises a skin-contacting mounting surface adapted for application to the skin of a subject by adhesive means, and with the infusion needle arranged such that in a situation of use it projects from the mounting surface to thereby penetrate the skin of the subject, whereby the place where the needle penetrates the skin is covered while the appliance is in use.

When a fluid drug is supplied to a user, it is important that the user can visually inspect the drug to make sure that the drug is not crystallised or polymerised due to e.g. self association or penetration, or that any other visually detectable change of the drug has occurred, such as oxidation of the active drug. For insulin such visual changes are often referred to as “fibrillation”. Even weak degrees of fibrillation can be critical for a patient, as it can potentially cause allergy to insulin and change the time-profile for the insulin. In practice, however, it is relatively difficult to observe weak fibrillation even in a cartridge under good lighting conditions.

Having regard to the above-identified prior art devices, it is an object of the present invention to provide a drug delivery device comprising or adapted to comprise an enclosed amount of a liquid drug with means aiding a user to check the condition of the drug, e.g. to check insulin for fibrillation. It is a further object of the invention to provide a device which allows a condition of a drug to be checked in an efficient way.

### DISCLOSURE OF EMBODIMENTS OF THE INVENTION

In the disclosure of embodiments of the present invention, embodiments and aspects will be described which will address one or more of the above objects or which will address objects apparent from the below disclosure as well as from the description of exemplary embodiments.

Thus, in a first aspect, a drug delivery device is provided, comprising a reservoir adapted to contain a liquid drug, an expelling assembly for, in a situation of use, expelling a drug from the reservoir. The device further comprises a light conductor having a light inlet and a light outlet, the light conductor being adapted for conducting light from a point of entrance and into the reservoir, and a transparent area allowing a user or external equipment to inspect a portion of the reservoir to thereby detect a transmission characteristic of the light through the drug. If external detection equipment is used the light need not be visible to the human eye. In this way an external light source can be used to effectively lighten the reservoir for inspection, just as the light conductor can be used to direct external light from a convenient point of entrance to the reservoir. However, a light conductor may also be used to route light generated within the device, e.g. from a LED arranged at a convenient location. The light conductor may be straight or adapted to conduct light in a non-straight manner, e.g. it may be flexible or it may comprise facets redirecting or deflecting light within the conductor. In the present context the term light conductor also covers the terms light guide and light pipe.

The transparent area may be formed solely by the reservoir per se, e.g. the user will directly observe a transparent portion of the reservoir, or a transparent portion of the reservoir may be covered by a further transparent member, e.g. a window arranged in a housing in which the reservoir is arranged. Although the transparent area(s) literally will conduct light, such structures merely serving as transparent barriers are not considered “light conductors” in the context of the present invention. In preferred embodiments a light conductor is arranged outside the visual path by which the user will inspect the reservoir.

The transmission characteristic may be any characteristic suitable of (i) being influenced by a relevant non-constant characteristic of the drug (e.g. fibrillation) and being detectable by either the user or detection means external to the device. For example, light focused or diffuse light would be dispersed in fibrillated insulin, the dispersion (at a given level) being visually identifiable by the user or other detection means.

The light inlet may be arranged to receive light generated external to the device, or the device may comprise a light generating source associated with the light inlet. Thus the light used my principally be of two different kinds, either light detectable by the human eye or light not detectable by the human eye, just as both kinds may be used in combination. In case the device is adapted for visual inspection by a user, visible light is used in combination with a transparent area allowing the user to inspect the portion of the device through which the light beam is directed. In case the device is adapted to be used with external detecting means sensible to the emitted light (e.g. a light sensor), the light may be either visible or non-visible to the human eye. Indeed, the same visible beam of light may be used for both purposes.

One or more light conductors may be arranged to substantially illuminate the interior of the reservoir, or one or more light conductors may be adapted to direct one or more beams of light through the reservoir.

Before turning to examples of reservoirs and pump arrangements in which the above disclosed embodiments, a different aspect of the invention will be described.

Thus, in a further aspect, a drug delivery device is provided, comprising a reservoir adapted to contain a liquid drug, and expelling means for, in a situation of use, expelling a drug from the reservoir. The device further comprises lighting means adapted for directing light through the drug, and detection means allowing a transmission characteristic of the light through the drug to be detected.

The transmission characteristic may be any characteristic suitable of (i) being influenced by a relevant non-constant characteristic of the drug (e.g. fibrillation) and being detectable by either the user or detection means incorporated in the device. For example, light focused or diffuse light would be dispersed in fibrillated insulin, the dispersion (at a given level) being visually identifiable by the user or other detection means.

In an exemplary embodiment the lighting means comprises a light conductor having a light inlet and a light outlet, the light conductor being adapted for conducting light from a point of entrance and into the reservoir, and wherein the detection means comprises a transparent area allowing a user to inspect a portion of the reservoir. In this way an external light source can be used to effectively lighten the reservoir for inspection, just as the light conductor can be used to direct external light from a convenient point of entrance to the reservoir. However, a light conductor may also be used to route light generated within the device, e.g. from a LED arranged at a convenient location. The light conductor may be straight or adapted to conduct light in a non-straight manner, e.g. it may be flexible or it may comprise facets redirecting light within the conductor. In the present context the term light conductor also covers the terms light guide and light pipe.

One or more light conductors may be arranged to substantially illuminate the interior of the reservoir, or one or more light conductors may be adapted to direct one or more beams of light through the reservoir.

The reservoir may comprise first and second flexible foil members sealed together to form a reservoir having a pouch-like configuration defining a general plane, the reservoir having a rounded edge portion. For such a reservoir, light may be directed through the rounded edge portion which will have a portion arranged substantially perpendicular to the general plane.

In a further exemplary embodiment the lighting means comprises light generating means adapted for directing a beam of light through the drug, the detection means allowing a trans-mission characteristic of the light beam through the drug to be detected.

The beam of light may be directed through a portion of the reservoir, or in case the device comprises a passageway arranged between the reservoir and an outlet, the beam of light being directed through a portion of the a passageway. Such a passageway may be a trans-port conduit between the reservoir and the pump, or it may be a portion of the expelling means, e.g. a conduit or space formed as part of a pump. The passageway may comprise a cavity associated with the detecting means and be adapted for detection purposes. Using a cavity or another portion of the passageway adapted for detection purposes would allow the drug to be inspected also when the reservoir is fully or partly concealed from view (e.g. when enclosed in a pump), or it may allow a more accurate determination of the relevant drug characteristic as would be possible with the reservoir.

The light used my principally be of two different kinds, either light detectable by the human eye or light not detectable by the human eye, just as both kinds may be used in combination. In case the device is adapted for visual inspection by a user, visible light is used in combination with detection means in the form of a transparent area allowing the user to inspect the portion of the device through which the light beam is directed. For such an application the device is advantageously provided with a switch allowing the user to turn the light beam on at demand. In case the device is provided with detecting means sensible to the emitted light (e.g. a light sensor), the light may be either visible or non-visible to the human eye. Indeed, the same visible beam of light may be used for both purposes.

In an exemplary embodiment the detection means comprises control means, light detection means adapted to detect light from the light generating means and transmit a signal indicative of a characteristic of the detected light to the control means, wherein the control means is adapted to identify a first signal range indicative of a first condition of a given drug, to identify a second signal range indicative of a second condition of the drug, and to generate a signal when the second condition has been identified. For example, a first range may be associated with non- or only minimally fibrillated insulin, and a second range may be associated with fibrillated insulin above a pre-determined level.

The device may also comprise a plurality of light detection means, each light detection means being adapted to detect light from the light generating means and transmit a signal indicative of a characteristic of the detected light to the control means, wherein the control means is adapted to identify on the basis of the transmitted signals a first and a second condition for a given drug, and to generate a signal when the second condition has been identified. By relying on more than one sensor a more robust and/or sensitive detection may be provided.

The light generating means may be formed integrally with the reservoir or a passageway, or it may be arranged external to a structure in which the drug is enclosed, the structure comprising a transparent portion allowing the light beam to enter the structure.

Irrespective of the actual inspection or detection arrangement, the reservoir may comprise first and second flexible foil members sealed together to form a reservoir having a pouch-like configuration defining a general plane, the reservoir having a rounded edge portion. For such a reservoir, light may be directed through the rounded edge portion which will have a portion arranged substantially perpendicular to the general plane.

When a flexible reservoir formed from foil material is provided, the expelling means may be in the form of a suction pump having, in a situation of use, a pump inlet in fluid communication with the reservoir.

The reservoir may also be in the form of a cylindrical member with a piston slidably arranged there within, i.e. corresponding to the kind of cartridge typically used for pen injection devices and infusion pumps. Such a reservoir may be either user-fillable or prefilled, just as it may be insertable into a drug delivery device or formed integrally therewith. Such a device would typically comprise a piston actuator, an electronic controller for controlling the piston actuator, and an energy source, e.g. corresponding to a typical, durable infusion pump.

Irrespective of the type of reservoir and the type of expelling means, in exemplary embodiments a drug delivery device of the invention may comprise a lower surface adapted for application towards the skin of a subject and a transcutaneous device adapted to penetrate the skin of the subject. An outlet of the delivery device may be adapted to cooperate with an infusion set (e.g. corresponding to a typical infusion pump), or the device may be provided with a transcutaneous access device, i.e. a needle, a soft cannula, a micro needle array or non-invasive transdermal means, projecting from or arranged on a lower surface of the device in a situation of use, such a device comprising an adhesive lower surface.

However, the principles of the present invention may also be implemented in a “pen” type delivery device, such a device comprising a dose setting member moveable to a selected set position representing a set dose of drug to be delivered, and a user actuation member allowing the set dose to be expelled. The expelling means may be purely mechanical or a motor may be used to drive the expelling means.

The term outlet is used to denote a structure which will serve as an outlet during actual delivery of drug from the delivery device. In other words, the outlet may be closed when not actually used. For example, the outlet may be in the form of a needle-penetratable septum which will be closed until a needle is arranged there through. The outlet may also be provided with a valve which will close the outlet until the delivery expelling means is actuated.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with references to the drawings, wherein

FIG. 1 shows in perspective view an embodiment of a modular drug delivery device,

FIG. 2 shows in an exploded perspective view a reservoir unit,

FIG. 3 shows in partial a reservoir unit comprising light conductors,

FIG. 4 shows in partial a further reservoir unit comprising light conductors,

FIG. 5 shows a schematic representation of a delivery device,

FIG. 6 shows a delivery device of the pen-type,

FIG. 7 shows a schematic representation of a further delivery device,

FIGS. 8A-8E show schematically embodiments of a reservoir unit comprising a light source, and

FIG. 9 shows the result of an experiment illustrating an aspect of the present invention,

In the figures like structures are identified by like reference numerals.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

Firstly, with reference to FIG. 1 an embodiment of a modular drug delivery device will be described. The delivery device is shown as an example of a type of device in which the present invention advantageously may be implemented, however, the present invention may be used in combination with any drug delivery device in which it is desirable to check a characteristic of the drug.

The transcutaneous device unit 2 comprises a transcutaneous device in the form of a needle and will thus in the following be termed a needle unit.

More specifically, FIG. 1 shows a perspective view of medical device in the form of a modular skin-mountable drug delivery device 1 comprising a patch-like needle unit 2 and a reservoir unit 5. When supplied to the user each of the units are preferably enclosed in its own sealed package (not shown).

The needle unit comprises a base portion 10 with a lower mounting surface adapted for application to the skin of a user, and a housing portion 20 in which a hollow infusion needle (not shown) is arranged. The needle comprises a distal portion adapted to penetrate the skin of a user, and a proximal portion adapted to be arranged in fluid communication with the reservoir unit. The distal portion of the needle is moveable between an initial position in which the distal end is retracted relative to the mounting surface, and an extended position in which it projects relative to the mounting surface. Further, the needle is moveable between the extended position in which the distal end projects relative to the mounting surface, and a retracted position in which the distal end is retracted relative to the mounting surface. The needle unit further comprises user-gripable actuation means in the form of strip-members 21, 22 for actuating respectively retracting the needle. The housing further comprises user-actuatable male coupling means 40 in the form of a pair of resiliently arranged hook members adapted to cooperate with corresponding female coupling means on the reservoir unit, this allowing the reservoir unit to be releasable secured to the needle unit in the situation of use. The base portion comprises a relatively rigid upper portion 11 attached to a more flexible adhesive sheet member 12 having a lower adhesive surface providing the mounting surface per se, the adhesive surface being supplied with a peelable protective sheet. The base portion also comprises a ridge member 13 adapted to engage a corresponding groove on the reservoir unit.

The reservoir unit 5 comprises a pre-filled reservoir containing a liquid drug formulation (e.g. insulin) and expelling means in the form of an electronically controlled pump for expelling the drug from the reservoir through the needle in a situation of use. The reservoir unit has a generally flat lower surface adapted to be mounted onto the upper surface of the base portion, and comprises a protruding portion 50 adapted to be received in a corresponding cavity of the housing portion 20 as well as female coupling means 51 adapted to engage the corresponding hook members 31 on the needle unit. The protruding portion provides the interface between the two units and comprises a pump outlet and contact means (not shown) allowing the pump to be started as the two units are assembled. The lower surface also comprises a window (not to be seen) allowing the user to visually control the contents of the reservoir, however, such a window may also be arranged on an upper free surface of the reservoir unit.

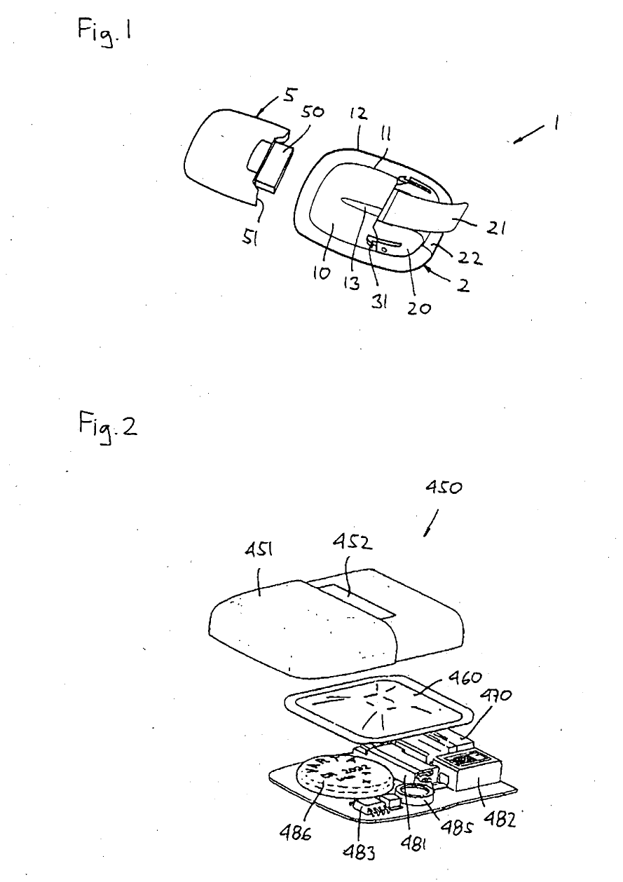

With reference to FIG. 2 an embodiment of a reservoir unit 450 of a type suitable to be used with the above described needle unit is shown or which may comprise the reservoir/pump portion of a unitary device, the reservoir unit comprising a housing 451 in which a flexible foil reservoir 460, a pump unit 470 in the form of a mechanically actuated membrane pump, and control and actuation means therefore are arranged. The housing comprises a display window 452 which may be used to provide information to the user, e.g. the amount of drug left in the reservoir or information in respect of a malfunction condition, and/or allow the user to visually inspect the contents of the reservoir. The control and actuation means comprises a pump actuating member in the form of a lever and piston arrangement 481 driven by a coil actuator 482, a microprocessor 483 for controlling the different functions of the reservoir unit, signal generating means 485 for generating an audible and/or tactile signal, and an energy source 486. The different components are arranged on a printed circuit board (PCB) 480. In the shown embodiment the reservoir is arranged above the PCB, however, alternatively it may be arranged below the PCB in combination with an inspection window arranged on a lower surface of the reservoir unit.

Different embodiments of the present invention may be embodied in the reservoir unit 450. For example, a light source (not shown) may be arranged on the PCB, the light source directing a beam of coloured light across a portion of the reservoir visible through the window 452. The width of the light beam may be chosen to allow the beam of light through non-fibrillated insulin to be invisible for the eye of the user, i.e. so narrow that it for practical purposes will not be observed. In this way a non-fibrillated insulin will result in the indication being “off”. However, if the insulin is fibrillated the light will get dispersed as it interacts with fibrils in the insulin, this dispersion scattering the light to make it visible for the user, potentially warning against further use of the insulin. Indeed, a given amount of fibrillation will be necessary in order to produce visible dispersion of the light, however, it has been found (see below) that dispersion of light is visible to the untrained eye before fibrillation becomes visible.

Depending on the type of light source it may be provided with a lens for focusing the light to produce a light beam. The light per se may e.g. be generated by a laser diode or a normal LED. As appears from the above-described working principle, the transmission of light through a wall portion of the reservoir should not in itself produce a noticeable amount of dispersion. Accordingly, for a flexible reservoir it may be desirable to attach a specialized “light guide” to the reservoir to properly guide light through the foil material without dispersion, or optical silicone may be used to bridge a gap between the light source and the reservoir.

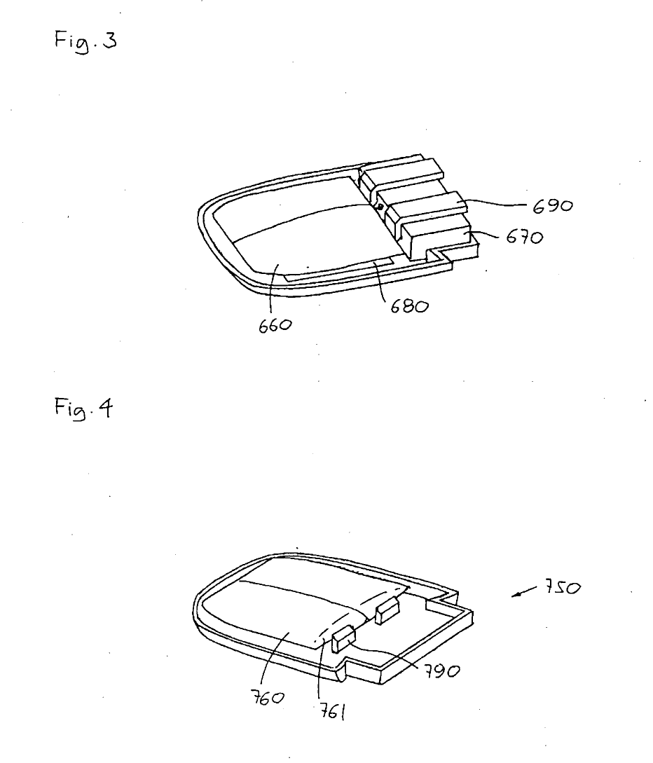

FIG. 3 shows a reservoir unit 650 with a portion of the housing removed. The reservoir unit comprises a pump unit 670, a flexible foil reservoir 660, and two light conductors 690. The reservoir comprises first and second flexible foil members sealed together at three edges to form a reservoir having a pouch-like configuration defining a general plane, the reservoir having a rounded edge portion (see FIG. 4). The lower surface of the reservoir unit is adapted to face towards a skin surface in a condition of use and is provided with a transparent area 680 allowing a user to inspect a portion of the reservoir to thereby detect a transmission characteristic of the light through the drug. Corresponding to the FIG. 2 embodiment, the pump unit further comprises a PCB (not shown) with additional components.

Each light conductor comprises a straight inlet portion with a light inlet end, and a straight outlet portion with a light outlet end, the two portions being arranged at an angle relative to each other. The inlet end may form a portion of the outer surface of the reservoir unit just as a light conductor may be formed integrally with other elements, e.g. a housing portion. Between the two portions and at the outlet end facets are provided for conducting light between the portions and out through the outlet end and into the reservoir in a direction substantially corresponding to the general plane of the reservoir. Optical silicone may be used to bridge a gap between the light conductor and the reservoir. In a situation of use the user will orient the light inlet towards a light source, e.g. a lamp, whereby light is conducted to the reservoir allowing the user to inspect the contents through a window in the housing (not shown) arranged on a surface of the reservoir unit or a corresponding device. The conductor may be formed to provide either a diffuse illumination of the reservoir or to provide two beams of light.

FIG. 4 shows in partial an alternative embodiment of a reservoir unit 750 in which a reservoir 760 with a rounded edge portion 761 is positioned on top of a PCB. Two light conductors 790 are arranged to conduct light from the lower surface of the unit and to the reservoir. The outlet ends of the light conductors are provided with facets for conducting light into the reservoir in a direction substantially corresponding to the general plane of the reservoir.

Instead of detecting fibrillation of a drug when located in the reservoir, detection may alternatively take place at a different location outside the reservoir. Such a location would typically be downstream of the reservoir as the drug is expelled from the reservoir towards the outlet. In this way a specialized detection structure can be provided which will not rely upon the inherent properties of a given reservoir. For example, it may be complicated to direct a beam of light through a portion of a flexible reservoir or through the wall of a stiff cartridge as in a conventional glass cartridge. Further, for a flexible reservoir the form of the reservoir will change as it is emptied. In contrast, a specialized structure arranged downstream of the reservoir can be adapted for the sole purpose of optimizing the detection of a desired drug property, e.g. fibrillation in insulin.

Correspondingly, in an exemplary embodiment of the invention a passageway is provided between the reservoir and the outlet, the light generating means being adapted for directing a beam of light through the drug in the cavity, and the detection means being adapted for detection of a drug transmission characteristic of the light beam through the drug in the cavity. The cavity may have any suitable configuration, e.g. formed as a cuvette, a channel, or a conduit.

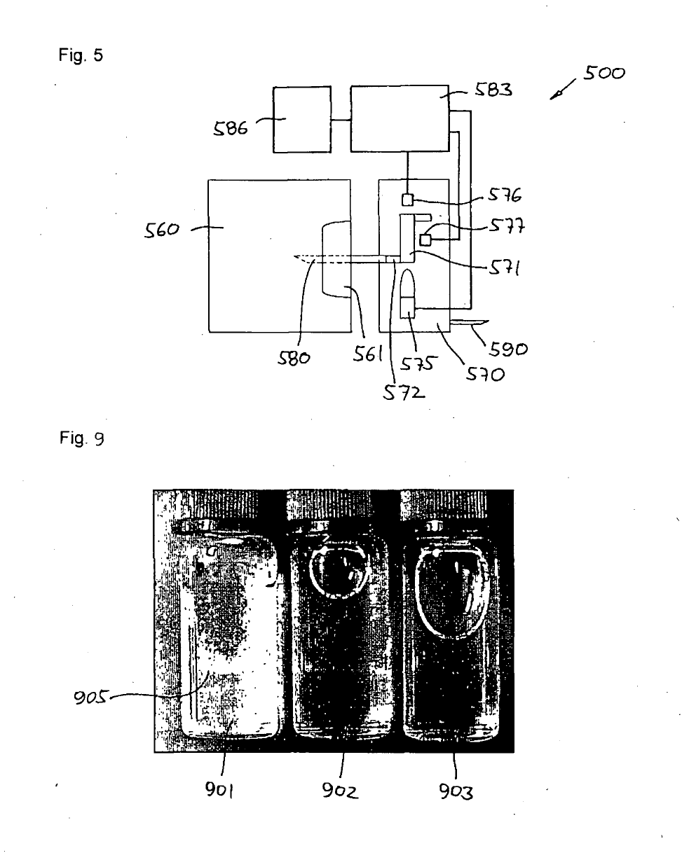

FIG. 5 shows in a schematic representation an embodiment of a delivery device 500 of the same general type as shown in FIG. 2. The delivery device comprises a flexible foil reservoir 560 including a needle-penetratable septum 561 welded onto the exterior surface of the reservoir, and a pump unit 570 including a membrane pump (not shown), a passageway 572 with cuvette 571, a light source 575 for producing a beam of light and light sensors 576, 577, as well as an energy source 586 and a processor 583 for controlling the pump, the light source and the sensors. The cuvette has an inlet 572 in fluid communication with a needle 580 arranged through the needle septum and thus in fluid communication with the reservoir, and an outlet 573 in fluid communication with the pump (not shown), the pump having an outlet in fluid communication with a transcutaneous access device 590, e.g. a needle.

If the device is adapted for visual inspection the upper side of the cuvette will be transparent to provide a window through which a user can inspect the interior of the cuvette and thus the beam of light there though. If the drug in the cuvette does not comprise any constituent which will disperse the light, the light beam is adapted to be invisible to the human eye, e.g. a narrow and well-defined beam of light as from a laser diode, this irrespective of the colour, e.g. red, of the light. When the drug changes a given characteristic, e.g. insulin starting to fibrillate, the light will get dispersed and will thus become visible to the human eye, see example below. In case only user detection is desired the sensor can be dispensed with.

When one or more light detecting sensors are provided, it will be possible to automatically detect a desired property of the drug. More specifically, a sensor may be provided to detect light from the light generating means and transmit a signal indicative of a characteristic of the detected light to the control means. For example, the sensor 577 may be arranged at the side of the cuvette to detect scattered light when a beam of light is transmitted through the drug. When e.g. the drug is fibrillated to a certain degree, the light beam will get dispersed and light will reach the sensor, this being indicative of a certain amount of fibrillation (or another characteristic of a drug). Correspondingly, the controller is adapted to identify a first signal range indicative of a first condition of a given drug (e.g. no or a low level of fibrillation), to identify a second signal range indicative of a second condition of the drug (e.g. an undesired level of fibrillation), and to generate a signal when the second condition has been identified. The signal may be used to alert the user of the condition and/or stop the pump.

When two or more light detectors are used (e.g. detecting scattered respectively transmitted light), the control means will be adapted to identify on the basis of the transmitted signals from the sensors a first and a second condition for a given drug, and to generate a signal when the second condition has been identified. Indeed, the control means may be adapted to detect more than two conditions.

By providing automatic detection, it will be possible to continuously detect the condition of the pumped drug without involving the user. This can be compared with the normal mode of operation of a drug delivery device in which the user is informed to check the condition of the drug to be infused prior to use. By continuously checking the condition of the drug, changes to the drug after it was transferred to the device or after a prefilled device was taken into use is possible. For example, if the detection means is arranged after the pump it will be possible to detect deterioration of the drug caused by the pumping it through the pump. Indeed, both automatic and user detection may be provided as shown in the FIG. 3 embodiment.

FIG. 6 discloses a further drug delivery device 100 suitable for incorporation of the present invention. More particularly, the device is a pen-type injection device adapted for discrete subcutaneous injection of user-set doses of a drug. Such a device may be either manually or motorized set/actuated. As appears, the device has a box-formed shape this making the device more suitable for implementation than a more traditionally formed long and slim pen. The delivery device 150 comprises a dose setting member 171, an actuation member 175, and a window 180 allowing the user to inspect a longitudinally arranged transparent cartridge. In accordance with an aspect of the invention, a light source is arranged within the delivery device to produce a beam of light across a distal portion of the cartridge (see FIG. 7). To turn the light source on a key 172 is provided. Alternatively a light conductor may be implemented to conduct light through a portion of the reservoir. In the shown embodiment the device is further provided with a detachable cap 190.

FIG. 7 shows in a schematic representation the interior of a semi-motorized version of a delivery device 200 of the type shown in FIG. 6. More specifically, a cartridge 201 with a piston 202 is accommodated in a not shown housing. In a situation of use a needle will be connected at the distal end of the cartridge. The piston is moved by a piston rod 203 which is shown as a threaded rod and forms a first dose setting element. A second dose setting element is a nut 204 with an internal thread co-operating with the external thread of the piston rod 203. A hollow member with a bore accommodating a proximal end of the piston rod forms an integral part of the nut to serve as an injection button 205. A pin 217 in the housing engages an axial track in the piston rod to block this piston rod against rotation relative to the housing.

When a dose is going to be set, the device is in an initial position with the nut 204 abutting a stop 206. The dose is set by activating one of a pair of dose setting keys 207 and 208 for counting forward respectively backward. The set dose is stored in an electronic controller 209 and is displayed on a display 210. As long as the counting up key is pressed the set dose is increased and the size of the dose may currently be followed on the display. If the counting up runs too far, the count down key may be activated until the set dose is decreased to the size wanted.

The mechanical setting of the dose setting elements according to the electronically set dose is performed by an electric motor 211 having an output shaft provided with an elongated gear 212 which gear engages a toothing 213 at the periphery of the nut 204. The motor may be controlled to run as well clockwise as anticlockwise and is controlled from the electronic circuit to rotate the nut in accordance with the set dose. The rotation is measured by a position reader 216 which counts the number and direction of passing teeth. When the dose has been set and the injection button 205 is depressed fully by the user the set amount of drug is expelled. Indeed, instead of using the motor to set a mechanically activated actuation button, the motor may be used to directly drive the piston rod in accordance with the set dose.

In accordance with an aspect of the invention, a light source 275 is provided for sending a beam of light through a distal portion of the cartridge, the beam (if visible) being viewable through a window in the housing. As shown, the device may further be provided with a detector 276 corresponding to the FIG. 5 embodiment. Alternatively a light conductor may be implemented to conduct light through a portion of the reservoir.

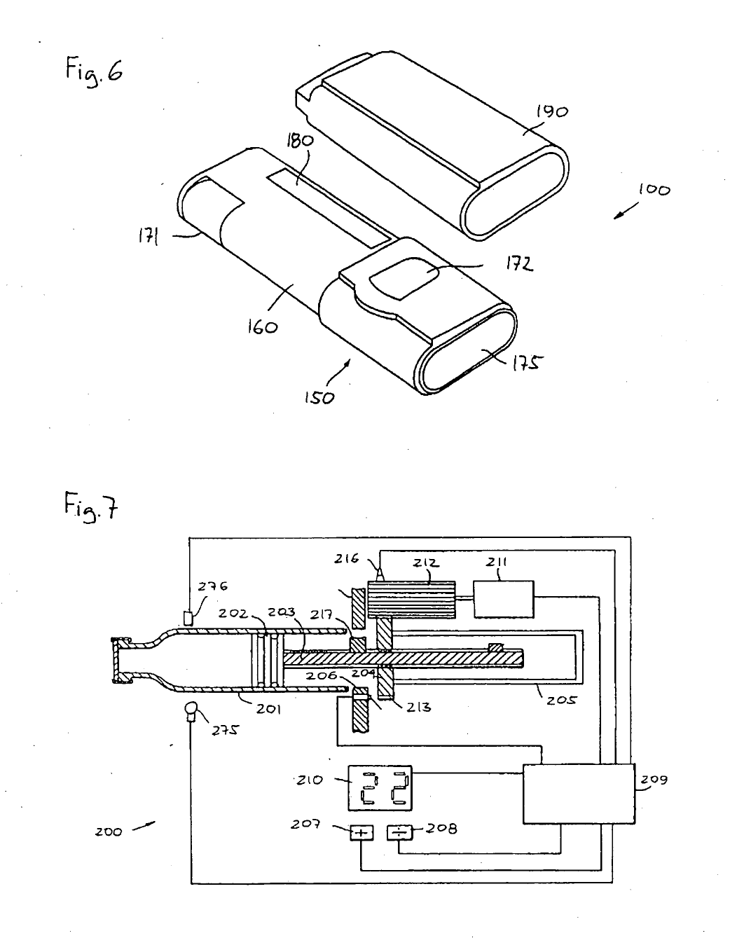

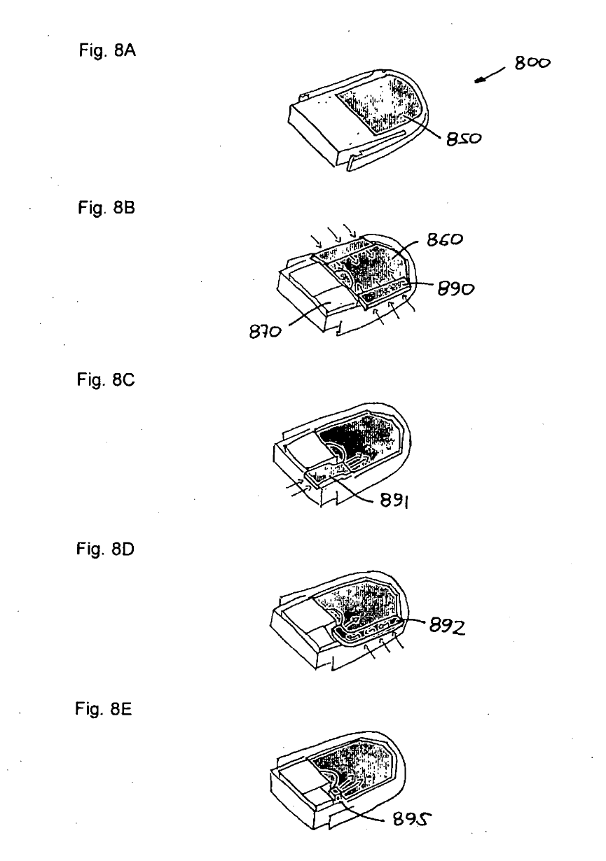

FIG. 8A shows a further embodiment 800 of a reservoir unit having a large transparent window 850 through which a reservoir is visible, and FIGS. 8B-8E show embodiments in which the housing portion with the window has been removed to disclose a flexible reservoir 860 and a pump assembly 870. FIG. 8B shows an embodiment in which light conductors 890 are arranged on each side of the reservoir conducting light from outside the unit to the reservoir, FIG. 8C shows an embodiment in which a light conductor 891 is arranged at an end surface of the unit, FIG. 8D shows an embodiment in which a bend light conductor 892 is arranged to conduct light from the side of the unit into the end of the reservoir, and FIG. 8E shows an embodiment in which a light source 895 is arranged within the unit to direct light into the reservoir. A light conductor may be arranged between the light source and the reservoir to conduct the light in a desired way into the reservoir.

FIG. 9 shows three cartridges with different degrees of fibrillated insulin, from strong fibrillation 901, over lightly, just recognizable fibrillation 902 to normal insulin 903. A red laser beam 905 is transmitted from right to left, but is only visible in the two cartridges with fibrillated insulin where dispersion of the light beam takes place.

The arrangements described above in accordance with the individual aspects of the invention can be used both independently of each other and in combination with elements in accordance with other aspects of the invention.

In the above description of the exemplary embodiments, the different structures providing mechanical, electrical and fluid contact and communication between the different components just as the means providing the described functionality for the different components (i.e. pump, reservoir, energy source, memory, control, display etc.) have been described to a degree to which the concept of the present invention will be apparent to the skilled reader. The detailed construction and specification for the different components are considered the object of a normal design procedure performed by the skilled person along the lines set out in the present specification.

1. A drug delivery device (650), comprising:
a reservoir (660) adapted to contain a liquid drug,
an expelling assembly (670) for, in a situation of use, expelling a drug from the reservoir,
a light conductor (690) having a light inlet and a light outlet, the light conductor being adapted for conducting light from a point of entrance and into the reservoir (660), and
a transparent area (452) allowing a user to inspect a portion of the reservoir to thereby detect a transmission characteristic of the light through the drug.

2. A device as in claim 1, wherein the light inlet is arranged to receive light generated external to the device.

3. A device as in claim 2, comprising a visible light generating source associated with the light inlet.

4. A device as in any of claim 1, wherein one or more light conductors are arranged to substantially illuminate the interior of the reservoir.

5. A device as in claim 1, wherein the light conductor is adapted to direct a beam of light through the reservoir.

6. A device as in any of claim 1, wherein the reservoir (690) comprises first and second flexible foil members sealed together to form a reservoir having a pouch-like configuration defining a general plane, the reservoir having a rounded edge portion through which the light is directed.

7. A device as in claim 5, wherein the expelling assembly comprises a pump having, in a situation of use, an inlet (580) in fluid communication with the reservoir.

8. A device as in any of claim 1, wherein the reservoir is in the form of a cylindrical member (201) with a piston slidably arranged there within.

9. A device as in claim 8, wherein the expelling assembly comprises a piston actuator, an electronic controller for controlling the piston actuator, and an energy source.

10. A device as in claim 1, wherein the light conductor is adapted to deflect incoming light to thereby direct it to the reservoir.

11. A device as in claim 1, further comprising:
a lower surface (12) adapted for application towards the skin of a subject,
a transcutaneous device adapted to penetrate the skin of the subject, the transcutaneous device being arranged in fluid communication with or being adapted to be arranged in fluid communication with the reservoir.

12. A device as in claim 11, wherein the expelling assembly comprises a dose setting member (171) moveable to a selected set position representing a set dose of drug to be delivered, and a user actuation member (175) allowing the set dose to be expelled.

13. A drug delivery device (500), comprising:
a reservoir (560) adapted to contain a liquid drug,
an expelling assembly (570) for, in a situation of use, expelling a drug from the reservoir,
a light source (575) adapted for directing a beam of light through the drug, and
detection means (576) allowing a transmission characteristic of the light beam through the drug to be detected.

14. A device as in claim 13, wherein the light is directed through a portion of the reservoir (201).

15. A device as in claim 13, comprising a passageway (572) arranged between the reservoir and an outlet, the beam of light being directed through a portion of the passageway.

16. A device as in claim 15, the passageway comprising a cavity (571), wherein the detection means (576) is associated with the cavity.

17. A device as claim 1, wherein:
at least a portion of the transmitted light is detectable by the human eye, and
the detection means comprises a transparent area allowing a user to inspect a portion of the device through which the light beam is directed through the drug.

18. A device as in claim 1, wherein the light generating means (575) is arranged external to a structure in which the drug is enclosed, the structure comprising a transparent portion allowing the light beam to enter the structure.

19. A device as in claim 1, wherein the detection means comprises:
control means (583),
one or more light detection means (575, 576), each light detection means being adapted to detect light from the light generating means (575) and transmit a signal indicative of a characteristic of the detected light to the control means, wherein
the control means is adapted to identify on the basis of the transmitted signal(s) a first and a second condition for a given drug, and to generate a signal when the second condition has been identified.

20. A device as in claim 1, wherein the reservoir is in the form of a flexible reservoir (560) formed from foil material.

21. A device as in claim 20, wherein the expelling assembly comprises a pump having, in a situation of use, an inlet (580) in fluid communication with the reservoir, and being connected to or comprising an outlet (590).

22. A device as in claim 1, wherein the reservoir is in the form of a cylindrical member (201) with a piston slidably arranged there within.

23. A device as in claim 22, wherein the expelling assembly comprises a piston actuator, an electronic controller for controlling the piston actuator, and an energy source.

24. A device as in claim 1, further comprising:
a lower surface (12) adapted for application towards the skin of a subject,
a transcutaneous device adapted to penetrate the skin of the subject, the transcutaneous device comprising an inlet in fluid communication with or being adapted to be arranged in fluid communication with the reservoir.

25. A device as in claim 23, wherein the expelling means comprising a dose setting member (171) moveable to a selected set position representing a set dose of drug to be delivered, and a user actuation member (175) allowing the set dose to be expelled.
